(19) United States
(12) Patent Application Publication
(10) Pub. No.: US 2013/0076898 A1
(43) Pub. Date: Mar. 28, 2013

(54) APPARATUS, SYSTEMS, AND METHODS FOR TRACKING MEDICAL PRODUCTS USING AN IMAGING UNIT

(71) Applicants: Richard Philippe, Laval (CA); Jonathan Assouline, Montreal (CA); Anders Larsson, Copenhagen (DK)

(72) Inventors: Richard Philippe, Laval (CA); Jonathan Assouline, Montreal (CA); Anders Larsson, Copenhagen (DK)

(21) Appl. No.: 13/559,158

(22) Filed: Jul. 26, 2012

Related U.S. Application Data
(60) Provisional application No. 61/513,721, filed on Aug. 1, 2011.

(51) Int. Cl. H04N 7/18

(52) U.S. Cl. CPC H04N 7/18
USPC 348/143

(57) ABSTRACT

According to one aspect there is an apparatus for tracking medical products. The apparatus includes a storage depot having a plurality of storage compartments, and an imaging unit having a first camera adapted to observe the storage compartments to track medical products therein.

### RELATED APPLICATIONS

This application claims the benefit of U.S. Provisional Patent Application Ser. No. 61/513,721 filed Aug. 1, 2011, and entitled APPARATUS, SYSTEMS, AND METHODS FOR TRACKING MEDICAL PRODUCTS USING AN IMAGING UNIT, the entire contents of which are hereby incorporated by reference herein for all purposes.

### TECHNICAL FIELD

Embodiments herein relate to apparatus, systems and methods for tracking medical products. More particularly, embodiments herein relate to apparatus, systems and methods for tracking medical products using an imaging unit that includes at least one camera.

### INTRODUCTION

Modern health care facilities can range from small and relatively simple medical clinics to large and complex hospitals. However, regardless of their size, health care facilities use many different types of medical products for treating patients depending on their health conditions. For example, medical products can include medications used to treat a particular ailment, implants inserted into a patient during a surgical procedure, as well as other supplies such as needles, gloves, syringes, etc.

Tracking the use and consumption of these medical products can be beneficial. For example, accurately tracking medical products can be helpful to ensure compatibility between different products (e.g. two medications) or for contacting patients when a product recall occurs. Tracking can also be useful for managing inventory to ensure that adequate supplies are ordered in a timely manner.

Traditionally, keeping track of medical products is challenging and is often performed manually. This usually requires a staff member to physically review inventory and fill out one or more forms with inventory details such as type of medical product, quantity consumed, quantity remaining, and so on. This manual review can be time consuming and can be error prone depending on the skill and attention of the staff member.

Accordingly, a need has been recognized for improved apparatus, systems and methods for tracking medical products at medical facilities.

### BRIEF DESCRIPTION OF THE DRAWINGS

For a better understanding of aspects of the apparatus, systems and methods described herein, and to show more clearly how they may be carried into effect, reference will be made to the accompanying drawings in which:

FIG. 1 is schematic side view of an apparatus for tracking medical products according to one embodiment;

FIG. 2 is another schematic side view of the apparatus of FIG. 1 showing a second camera;

FIG. 3 is an overhead view of a storage compartment of the apparatus of FIG. 1 having four storage regions;

FIG. 4 is a schematic of a system for tracking medical products according to one embodiment;

FIG. 5 is a schematic of a system for tracking medical products according to another embodiment having an image processing server;

FIG. 6 is a front elevation view of the apparatus of FIG. 1 in use with a user;

FIG. 7 is a screenshot of an apparatus adapted to detect facial information of a user;

FIG. 8 is a screenshot of an apparatus adapted to detect hand information of a user;

FIG. 9 is an image of examples of hand signals that may be suitable for use with the apparatus of FIG. 8;

FIG. 10 is a screenshot of an apparatus adapted to respond to hand signals of a user;

FIG. 11 is an image of a user's hand being tracked while removing an object from a storage compartment;

FIG. 12 is a front elevation view of the apparatus of FIG. 1 showing a user located beyond a second detection region;

FIG. 13 is an illustration of a medical product having a bar code thereon and a wireless communication device for use by the user;

FIG. 14 is an overhead view of a medical product having a bar code being added to a storage compartment;

FIG. 15 is an overhead view of a medical product being rotated between different secondary and primary regions within the storage compartment; and

FIG. 16 is an overhead view of the storage compartment of FIG. 15 after moving the medical product.

### DESCRIPTION OF VARIOUS EMBODIMENTS

It will be appreciated that numerous specific details are set forth herein in order to provide a thorough understanding of the exemplary embodiments as described. However, it will be understood by those of ordinary skill in the art that the embodiments described herein may be practiced without these specific details. In other instances, well-known elements, methods, procedures and components have not been described in detail so as not to obscure the embodiments described herein. Furthermore, this description is not to be considered as limiting the scope of the embodiments described herein in any way, but rather as merely describing the implementation of various embodiments as described.

In some cases, at least some elements of the apparatus, systems and methods described herein may be implemented in hardware or software, or combinations of both. Some elements may be implemented in computer programs executing on programmable computers which may include at least one processor, at least one data storage device (which may include volatile and non-volatile memory and/or other storage elements), at least one input device, and at least one output device. For example and without limitation, the programmable computers may include a mainframe computer, server, personal computer, laptop, personal data assistant, tablet computer or cellular telephone or smartphone, including one or more processors. Program code may be applied to input data to perform functions as described herein and generate output information. The output information may be applied to one or more output devices in known fashions.

Each program may be implemented in a high level procedural or object oriented programming and/or scripting language to communicate with a computer hardware system. In some embodiments the programs can be implemented in assembly or machine language, if desired. In any case, the language may be a compiled or interpreted language. Each such computer program may be stored on a storage media or a device (e.g. read only memory (ROM) or magnetic diskette) readable by a general or special purpose programmable computer, for configuring and operating the computer when the storage media or device is read by the computer to perform the procedures described herein. The apparatus, systems and methods may also be implemented in some embodiments as a non-transitory computer-readable storage medium, configured with a computer program, wherein the storage medium so configured causes a computer to operate in a specific and defined manner to perform at least some of the functions described herein.

Throughout the present specification, the expression “medical product” is used to generally refer to any type of product or material that may be used or administered in the treatment of patients. A non-exhaustive list of exemplary medical products could include medications, intravenous solutions, catheters, tubes, implants, pacemakers, gloves, needles, syringes, and so on.

Various techniques to track medical products have been developed. For example, U.S. patent application Ser. No. 12/578,683 entitled SYSTEM AND METHOD FOR TRACKING MEDICAL PRODUCTS IN A TWO BIN PER MEDICAL PRODUCT REPLENISHMENT SYSTEM (the entire contents of which are hereby incorporated by reference herein) describes storing medical product information (including a lot number and date of receipt), and recording replenishment requests and generated dates. RFID tags are affixed to bins containing medical bins, and location information of the corresponding RFID tags is detected by an RFID reader to determine probable locations for those medical products. This information may then be used for replenishment requests.

However, tracking medical products using RFID tags affixed to such bins may be undesirable. For example, the bins, the packaging of the medical products or the medical products themselves may need to be modified to include the RFID tags. Moreover, radio transmission interference from multiple RFID tags or due to ambient conditions around the RFID tags and/or RFID readers can lead to erroneous readings of the RFID tags such as missed reads or false positives. As such, RFID tags may not be suitable for tracking medical products in some circumstances.

In contrast to prior techniques, the teachings herein relate to the use of an imaging unit to track medical products. Each imaging unit includes one or more cameras adapted to visually inspect storage compartments (e.g. drawers in a cabinet, bins on open shelves, etc.) and determine the presence or absence of medical products therein based on one or more visual indicators.

The visual indicators could be a simple detection of whether the storage compartment is empty or contains some object without necessarily identifying that particular object.

Where an object is present, the visual indicators could further include shapes, patterns, and/or colors that are associated with particular medical products. For example, a library of medical product shapes, patterns and/or colors could be stored in a database and then compared with an observed object to determine what particular medical product has been observed (or a reasonable estimate thereof).

In some cases, the visual indicators could include bar codes (e.g. two-dimensional or three-dimensional bar codes) or other visible markings (e.g. letters, numbers, product names, trademarks etc.) imprinted on, affixed to, or otherwise associated with the medical products. For instance, a box of syringes may have a bar code thereon that can be scanned by the imaging unit. In other examples, a product name (e.g. Hamilton Syringe) could be listed on the packaging.

In some cases, the bar code or other visual indicator may include expiry information, a serial number, and/or other details about the medical product. In some embodiments, the visual indicator may be linked to such details about the medical product (e.g. via a product database).

In some embodiments, visual indicators may be used to communicate information or commands to the imaging unit. The information and commands may relate to the medical product or to other aspects of health care. For example, the visual indicator may be used to communicate a patient number or bed identification number so that when medical products are subsequently removed from the storage compartment, they can be associated with that particular patient in a patient database. This can be useful for ensuring that particular high-value items (e.g. a pacemaker) are associated with the correct patient. Some such visual indicators could include gestures made by a user, including facial gestures, and hand signals.

The use of a computerized imaging system tends to be beneficial in that it may provide for a “hands-off” system for use by health care personnel. This can reduce the need of the health care personnel to physically interact with input and/or output devices in a medical product tracking system. This can help avoid contamination and maintain a sterile environment, for example by eliminating the need to touch a keyboard, mouse, touchscreen, etc., which may be particularly desirable in a medical facility.

This system may also provide for automatic tracking of the picking, consumption and replenishment of medical products, capturing quantities and/or unique identifiers for tracking unique items. For instance, once a storage compartment has been organized, the consumption of particular medical products from that storage compartment can be automatically tracked. This information can then be used to initiate product replenishment based on inventory levels. For example, when the imaging unit detects that a secondary region or bin is empty in a “two-bin” replenishment system, the corresponding additional medical products can be ordered automatically, or by notifying appropriate individuals that an order should be placed.

There are several technical challenges that make implementing computerized imaging systems in a medical environment non-trivial. The first challenge is providing proper visibility and lighting for the cameras being used. To this end, the vision algorithms selected for use with the imaging unit should be able to recognize the presence, partial presence and/or absence of many different medical products in a variety of lighting conditions, including low lighting conditions. In some embodiments, the imagining units may include lights for illuminating the medical products and/or storage compartments to assist the camera(s) in obtaining good images.

Another challenge includes recognizing and properly interpreting user activities, such as a user approaching a storage compartment, opening a drawer, removing a medical product from the storage compartment, putting an item in the storage compartment (e.g. restocking a medical product), or changing the position of a medical product (e.g. rotating medical products from a secondary region of the storage compartment to a primary region in a “two-bin” system).

In some embodiments, the imaging units may be adapted to respond to gestures (such as hand signals) to initiate tracking of one or more of these activities. In some embodiments, the imaging units may be adapted to interpret user activities and determine the corresponding action with or without the use of gestures (e.g. determining whether a medical product is being removed from or added to a storage compartment).

Some further technical challenges may be specific to the particularities of how medical products are stored and used in the medical field. For example, the arrangement of storage cabinets or depots may be both beneficial and detrimental for computerized vision systems. In particular, in a medical facility the primary goal of a storage depot (e.g. a cabinet, rack, etc.) should be to enhance the effectiveness of medical personnel and as such medical products are often organized in an orderly and consistent manner. The orderly disposition of items tends to be an advantage for the imaging units, as the consistency lends itself well to recognizing medical products and particular behaviors.

However, the storage compartment arrangement may not be so favorable depending on the normal use by medical staff. For example, medical personnel will often reach inside a cabinet without completely opening a drawer, inhibiting cameras from getting a good view of the drawer's contents as a medical product is removed. Moreover, when a user moves quickly, it can be difficult for the imaging unit to properly interpret the user's actions.

Accordingly, the imaging units should adapted to react fast enough to recognize medical products that are in movement, in some cases with only a small amount of image detail. To this end, video cameras that are capable of capturing high resolution images at high frame rates may be particularly suited. Moreover, various different algorithms for image enhancement, image segmentation, pattern recognition and gestural recognition may assist with meeting at least some of these challenges.

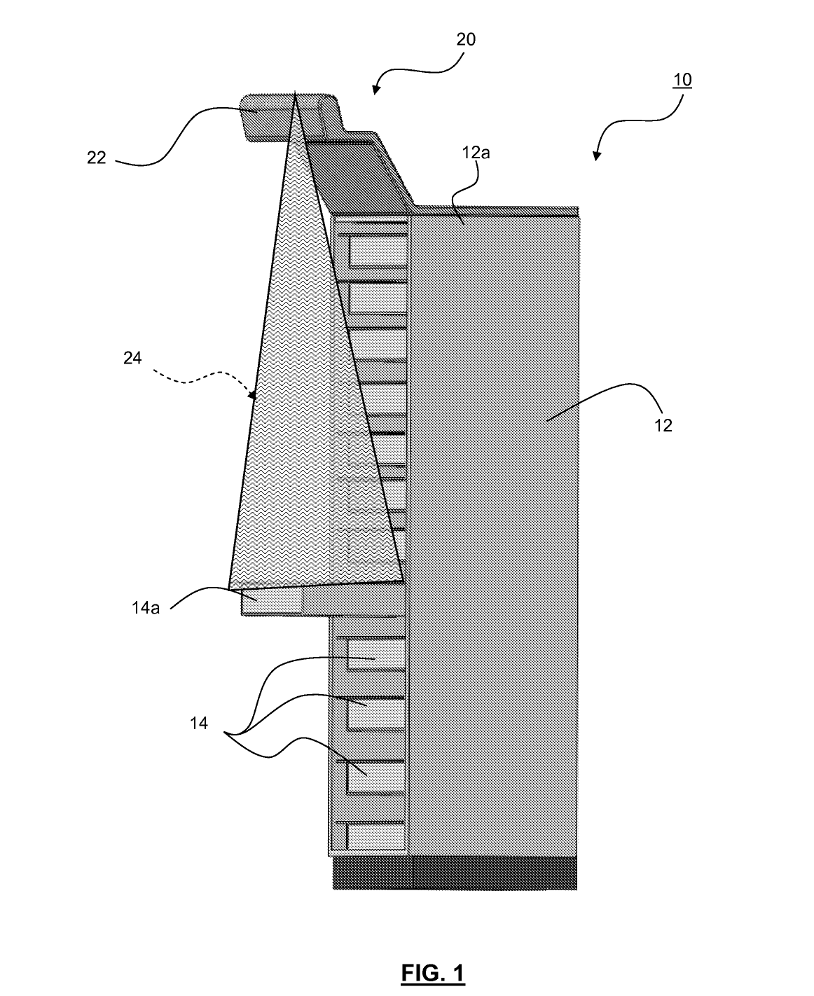

Turning now to FIG. 1, illustrated therein is an apparatus 10 for tracking medical products according to one embodiment. As shown the apparatus 10 includes a storage depot 12, which could be a cabinet, a rack, etc. Located within the storage depot 12 are storage compartments 14, which could be drawers, shelves, etc. For example, in the illustrated embodiment the storage depot 12 is a cabinet with a plurality of storage compartments 14 in the form of drawers, including a first drawer 14a shown in an “open” position that extends outwardly from the front of the cabinet 12.

The apparatus 10 also includes an imaging unit indicated generally as 20. The imaging unit 20 includes at least one camera 22 that is positioned and adapted to observe the storage compartments 14 to track medical products therein. In particular, the camera 22 has a detection region 24, which represents the region visible to the camera 22 and allows the imaging unit 20 to observe the contents of one or more storage compartments 14 (such as the open drawer 14a).

As shown, the camera 22 may be mounted at a top end 12a of the storage depot 12 and be orientated downwards such that the detection region 24 points downwardly toward the open drawer 14a.

It will be appreciated that the detection region 24 may vary in size and shape, particularly to accommodate different storage compartments 14, which may be opened and closed at different distances D from the camera 22. It will also be appreciated that in some embodiments the imaging unit 20 could be mounted at other locations on the storage depot 12 (e.g. on the side of a cabinet).

In some embodiments, the imaging unit 20 may be adapted to determine whether a particular storage compartment 14 (or a particular region of the storage compartment 14) is empty or not. For example, the imagining unit 20 may be operable to detect whether any object is located within the storage compartment 14 without necessarily identifying the type of medical product therein. In some embodiments this may be done by comparing a previous image of an empty storage compartment 14 with the image captured by the camera 22, and by checking for differences therebetween. In some embodiments, the storage compartments 14 may be of a uniform color (e.g. white) and the imaging unit 20 may detect color or shadow differences to determine whether an object is present.

In some embodiments, the storage compartment 14 may have a uniform flat surface, and the imagining unit 20 may detect distance variations on the flat surface as an indication that an object is present.

In some other embodiments, the imaging unit 20 may be adapted to determine not only whether an object is present, but what particular medical products are in the storage compartment 14. For example, shape, size and pattern recognition algorithms may be used to visually identify one or more particular medical products in one or more regions of the storage compartment 14. In some cases this may be done by comparing the observed images captured by the camera 22 to a database of known medical products.

In some embodiments, the imaging unit 20 may be adapted to detect a user's hand as it moves near the storage compartment 14. This may be useful for determining when a medical product is being added to or removed from the storage compartment 14. For example, the imaging unit 20 may track the motion of a hand and use motion tracking information (e.g. speed of the hand, changes of direction, and so on) as indicators to help determine which medical product has been added or removed.

In some embodiments, the camera 22 of the imaging unit 20 may include infrared capabilities which allow for the measurement of body heat. This may assist the imaging unit 20 in distinguishing a user's body part (e.g. a hand), which will normally be at an elevated temperature of 98.6 degrees Fahrenheit, from the storage compartments 14 or medical products therein, which will normally be at ambient temperatures (e.g. between about 65 degrees Fahrenheit to about 75 degrees Fahrenheit).

In some embodiments, the imaging unit 20 may be adapted to read a bar code, alphanumeric characters, or other optical markings associated with one or more medical products in the storage compartment 14 to help identify the particular medical products therein.

In some embodiments, the apparatus 10 may be adapted to detect which particular storage compartment (e.g. the drawer 14a) of a plurality of storage compartments is open. In some cases, this may be done, for example, by using the imaging unit 20 to read an identifier on the particular storage compartment 14 (e.g. the drawer 14a). The identifier on the storage compartment 14 may be for example a barcode, an alphanumeric identifier, a color pattern, and so on.

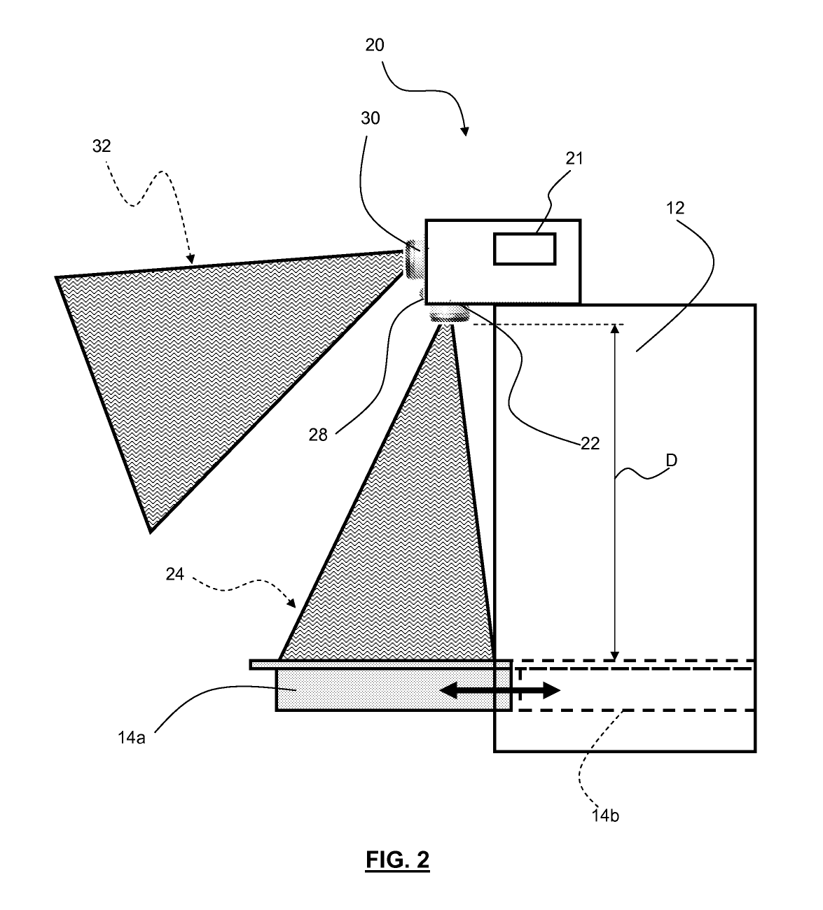

In some embodiments, the particular storage compartment 14 that is open may be determined by measuring the distance D (as shown in FIG. 2) between the camera 22 and the open storage compartment 14. This measured distance D can then be compared against predetermined distances for the storage compartments 14 to determine which particular storage compartment has been opened.

In some embodiments, sensors (e.g. Hall-effect sensors) positioned on the storage depot 12 may be used to identify which particular storage compartment 14 is open, although this may tend to increase the cost and complexity of the apparatus 10. For example, a Hall-effect sensor may detect the first drawer 14a as it moves from the closed position (indicated generally as 14b) to the open position shown in FIG. 2.

In some embodiments, the imaging unit 20 may also include a confirmation light 28 for communicating information to the user (such as the status of the storage depot 12, storage compartments 14, or medical products associated therewith).The confirmation light 28 may be an LED or another light, located at a position selected to be visible to a user. In some embodiments, the confirmation light 28 could include a display screen, such as an LCD screen.

The confirmation light 28 may communicate with the user based on one or more states (e.g. illuminated or off, blinking, displaying a particular color, and so on). For instance, the confirmation light 28 may be illuminated when one or more of the storage compartments 14 is open, or when a change of status is detected (e.g. a medical product has been added or removed from a storage compartment 14).

In some embodiments, the confirmation light 28 may display different colors to indicate different states or activities. For example, a first color (e.g. green) may be used to indicate that the open storage compartment 14 is full, or includes at least one medical product therein. A second color (e.g. red) may be used to indicate that a particular storage compartment 14 is empty. A third color (e.g. amber) may be used to indicate an intermediate state, or indicate an error has occurred (in some cases in combination with blinking), and so on.

In some embodiments, other techniques may be used to indicate states or communicate information to a user. For example, a speaker could be used to generate audible alerts (e.g. beeps) or express a recorded voice.

As shown in FIG. 2, in some embodiments the imaging unit 20 may include a second camera 30. As shown, the second camera 30 may be orientated outwardly to define a second detection region 32. The second camera 30 may be useful for augmenting or enhancing the detection and/or tracking functions of the imaging unit 20.

In particular, the second camera 30 may be useful for detecting the presence or absence of a user, for recognizing a particular user (e.g. using facial recognition techniques), and/or for detecting gestures made by the user (e.g. facial gestures such as a smile or a frown, and/or hand signals) which may be useful for controlling one of more aspects of the imaging unit 20. For example, a hand signal may be used to identify a particular user. A hand signal may also indicate that the user is accessing a particular storage compartment 14, adding or removing certain medical products in association with a specific patient, or has completed a task.

In some embodiments, two cameras (e.g. the cameras 22, 30 or other cameras) may be oriented in the same or a similar direction, which could assist with determining spatial depth by comparing the images between the two cameras. This may be particularly useful for determining hand signals as described below.

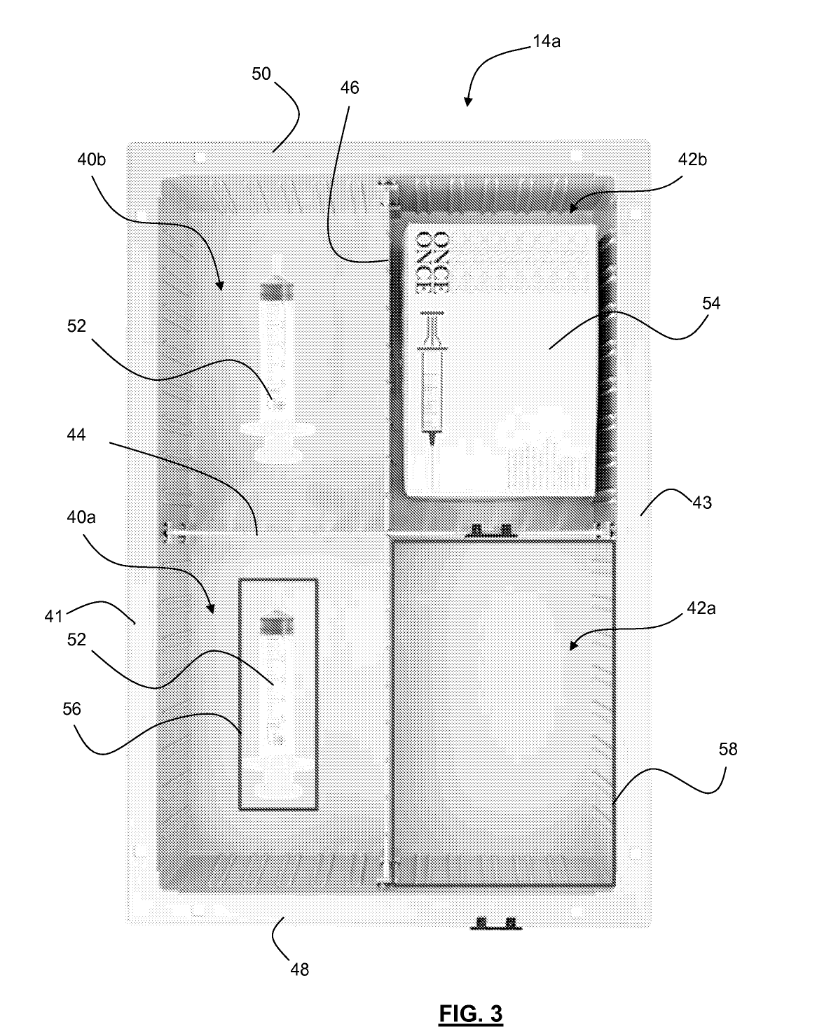

Turning now to FIG. 3, illustrated therein is an overhead view of the first drawer 14a according to one embodiment. In this embodiment, the first drawer 14a is divided into four storage regions 40a, 40b, 42a, 42b (which may also be referred to as “bins”). It will be understood that this is an example only and the number, arrangement, and configuration of the storage regions is not meant to be limiting. In particular, in some embodiments only one storage region may be present on a storage compartment 14. In other embodiments, two or more, and in particular four or more, storage regions may be provided on a particular storage compartment 14.

As shown in FIG. 3, the drawer 14a has a front 48 and a rear 50. Two storage regions 40a, 40b are located on a left side 41 of the drawer 14a, while two other storage regions 42a, 42b are located on a right side 43 of the drawer 14a. The storage regions 40a, 42a near the front 48 of the drawer 14a may be referred to as “primary” storage regions, while those regions 40b, 42b near the rear 50 of the drawer 14a may be referred to as “secondary” regions. In particular, since the primary regions 40a, 42a are closer to the front 48 of the drawer 14a, medical products therein will normally be picked first by a user. Once the primary regions 40a, 42a are empty, medical products from the secondary regions 40b, 42b can be moved or “rotated” into the primary regions 40a, 42a. The secondary regions 40b, 42b can then be resupplied, for example at some later time.

In some embodiments, the various storage regions 40a, 40b, 42a, 42b in a drawer 14a can be defined by one or more dividers 44, 46. In some embodiments the dividers 44, 46 may be movable and reconfigurable so that the layout of the storage regions 40a, 40b, 42a, 42b on a particular storage compartment 14 can be varied.

In other embodiments, the storage regions 40a, 40b, 42a, 42b may be provided as separate bins or baskets that are located on a shelf or drawer of a storage compartment 14.

In some embodiments, the imaging unit 20 may adapted to learn the particular layout of each particular storage compartment 14 (e.g. the size and shape of the regions 40a, 40b, 42a, 42b may be observed and stored in a database using the imaging unit 20 in a “learning” or training mode). The learning mode may also be adapted to allow for reconfiguration of storage compartment layouts.

The imaging unit 20 can also be configured to generate an alert if a particular storage compartment appears to be misconfigured (e.g. the dividers 46, 48 appear to be in the incorrect position based on the layout learned, or the wrong medical products have been detected in a particular storage compartment 14).

In the illustrated embodiment, a first type of medical product 52 (e.g. a syringe) is located within each of the storage regions 40a, 40b, a second type of medical product 54 (e.g. a box of needles) is located in the secondary storage region 42b, and the primary storage region 42a is empty.

During use the imaging unit 20 can define one or more detection zones 56, 58 around one or more of the medical products 52 (e.g. the detection zone 56 is around the syringe in storage region 40a) and/or within one or more regions 40a, 40b, 42a, 42b (e.g. the detection zone 58 is around storage region 42a). The detection zones 56, 58 allow the imaging unit 20 to determine whether there is an object in the corresponding region 40a, 40b, 42a, 42b. For instance, a medical product 52 is observed to be in the detection zone 56. Therefore, the imaging unit 20 can determine that an object is present in that primary storage region 40a. Similarly, the detection zone 58 in the primary storage region 42a is devoid of any objects, which indicates that the storage region 42a is empty.

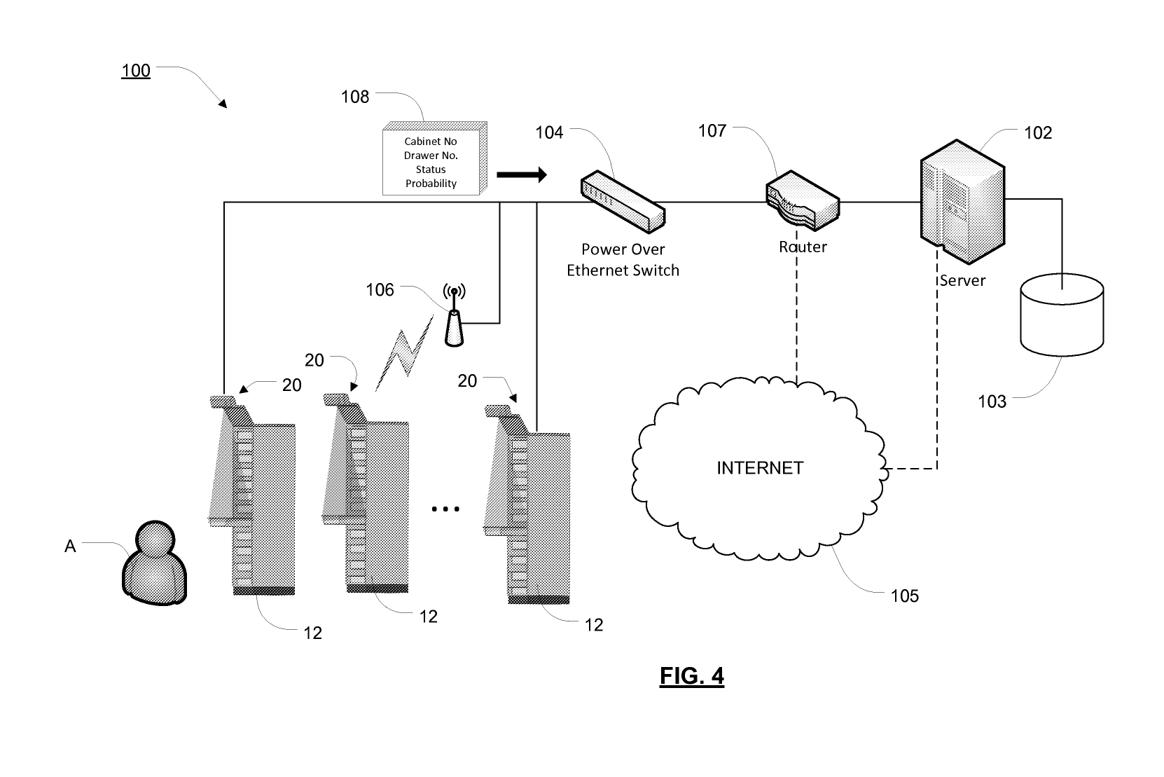

Turning now to FIG. 4, illustrated therein is a system 100 for tracking medical products according to one embodiment. As shown, the system 100 includes one or more storage depots 12 (e.g. cabinets). Each storage depot 12 has an imaging unit 20 associated therewith, which may include an imaging processor 21, cameras 22, 30 and other elements as generally described above.

The system 100 also includes at least one server 102. Generally the imaging unit 20 of each storage depot 12 is adapted to communicate with the server 102 so the system can track medical products consumed and/or replenished for each storage depot 12.

In some embodiments, the system 100 may also include a database 103, which can be in communication with the server 102. The database 103 may store various information, such as information about known medical products (e.g. shape, pattern, and color information), user information (e.g. facial recognition details for authorized users), patient information (e.g. which medical products have been used on which patent), and so on.

In some embodiments, the system 100 may also include one or more switches 104 or routers for routing information from the imaging units 20 to the server 102.

In some embodiments, one or more of the imaging units 20 may communicate with the server 102 over the Internet 105 (e.g. via a router 107). This may be useful, for example, when an imaging unit 20 and the server 102 are located at different physical locations. For example, the server 102 may be located in a separate building away from the medical facility which houses the storage depots 12 (e.g. at a medical product warehouse).

In some embodiments, one or more of the imaging units 20 may communicate with the server 102 using a wireless access point 106 (e.g. a Wi-Fi hotspot, a cellular or other wireless data communications channel). This may be particularly useful when a storage depot 12 is mobile (e.g. provided on wheels) as it need not be coupled to a wired connection.

In the system 100, the imaging units 20 include at least one imaging processor 21 (as shown in FIG. 2). The imaging processor 21 is adapted to process the image information obtained by the cameras 22, 30 and perform data analysis on the image information so that more specific processed data 108 can be sent to the server 102.

This processed data 108 may include, for example, information about a particular storage depot 12 (e.g. a cabinet number), information about a particular storage compartment 14 (e.g. a drawer number), information about a particular region 40a, 40b, 42a, 42b of a storage compartment 14, status information (e.g. a particular region 40a has or does not have an object therein), and/or medical product information (e.g. information about a particular medical product 52 located within a particular region 40a, which could include bar code information for that medical product 52, expiry information, and so on).

In some embodiments, the imaging processor 21 (shown in FIG. 2) may evaluate the quality of the information being sent to the server 102, which may be used to identify data errors. More particularly, the imaging processor 21 may be adapted to determine a “probability of certainty” for the processed data 108 sent to the server 102. For example, depending on the quality of the images received from the cameras 22 and 30 (shown in FIG. 2), the imaging processor 21 may have varying levels of confidence in the accuracy of the processed data 108.

Generally the processed data 108 may be used by the server 102 to make replenishing decisions, such as determining how much of a particular medical product to order for a medical facility.

For example, during use a user A may approach a particular storage depot 12 and open a drawer. The imaging unit 20 can then detect which drawer has been opened. In some embodiments, the imaging unit 20 may indicate to a user that the drawer is fully open (e.g. by flashing the confirmation light). The imaging unit 20 can then inspect the drawer, and report status information to the server 102 (e.g. the secondary region 42b is empty). The imaging unit 20 can also monitor the status of the drawer. For example, if the user A removes a particular medical product, this information can be sent to the server 102 (in some embodiments in real time or substantially real time, in other embodiments after the open drawer has been closed).

Providing an imaging processor 21 on the imaging unit 20 can make the imaging unit 20 “smart” in the sense that it is able to perform at least some image analysis locally. This can be beneficial, as the processed data 108 sent to the server 102 can be more precise and include only relevant or desired information, which may consume less bandwidth. This may be particularly useful for imagining units 20 that communicate using a wireless access point 106 where bandwidth may be limited. However, using an imaging processor 21 will generally require more processing to be performed at the imaging unit 20, which can increase the costs of the imaging unit 20 (as more a complex processor and more memory may be required). Use of the imaging processor 21 might also result in slower operation of the imaging unit 20 and may consume more power.

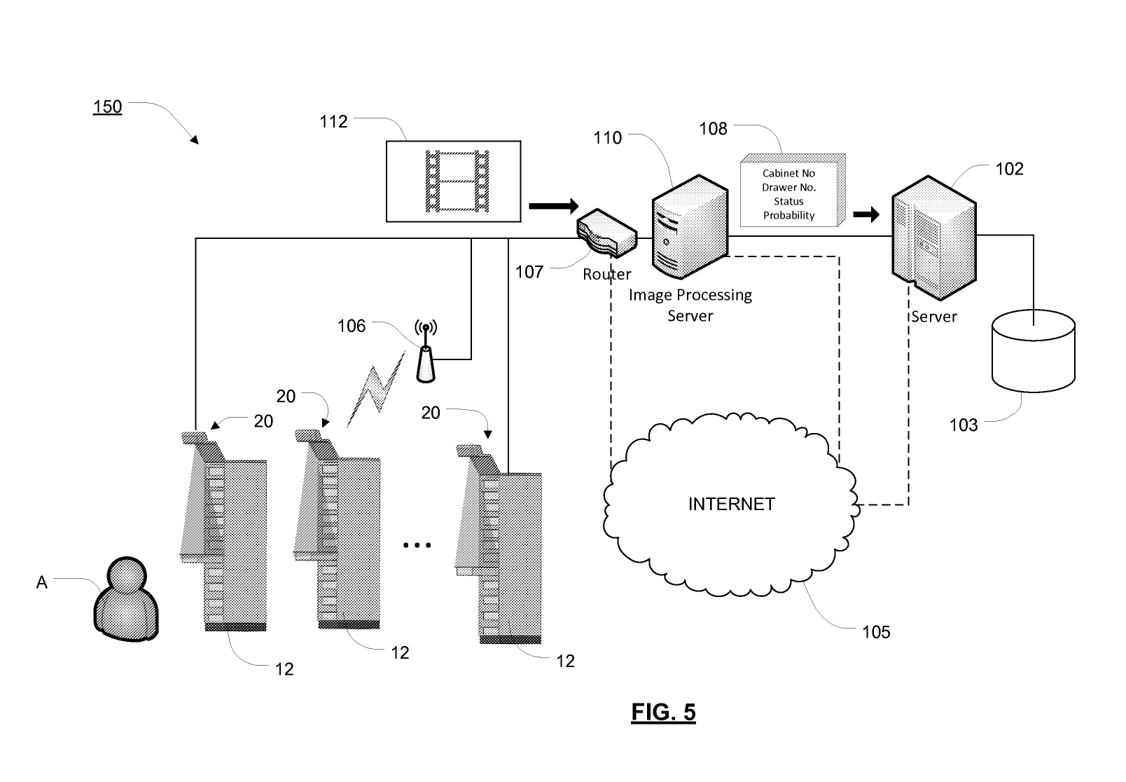

Accordingly, in some other embodiments (as shown in FIG. 5) a system 150 may include imaging units 20 that are generally not adapted to perform complex image analysis (e.g. “dumb” imaging units). In such embodiments the imaging units 20 may send raw image data 112 (e.g. a raw video stream) to an image processing server 110. The image processing server 110 can then analyze the raw image data 112 and send the processed data 108 (e.g. information about particular cabinets, medical products, etc.) to the server 102.

In some embodiments, the imaging units 20 may send their raw image data 112 to the image processing server 110 over the Internet 105.

The system 150 may be advantageous over the system 100 in some embodiments in that the imaging units 20 can be quite simple and need not be capable of performing complex imaging analysis. As a result, the cameras 22, 30 of the imaging units 20 in the system 150 may be webcams or other low-cost cameras. However, since such imaging units 20 may need to send significant quantities of raw image data 112 to the image processing server 110, the system 150 may consume large amounts of bandwidth. Moreover, the system 150 may be slower, particularly where large numbers of imaging units 20 are used, since the image processing server 110 can become a choke point for the system 150.

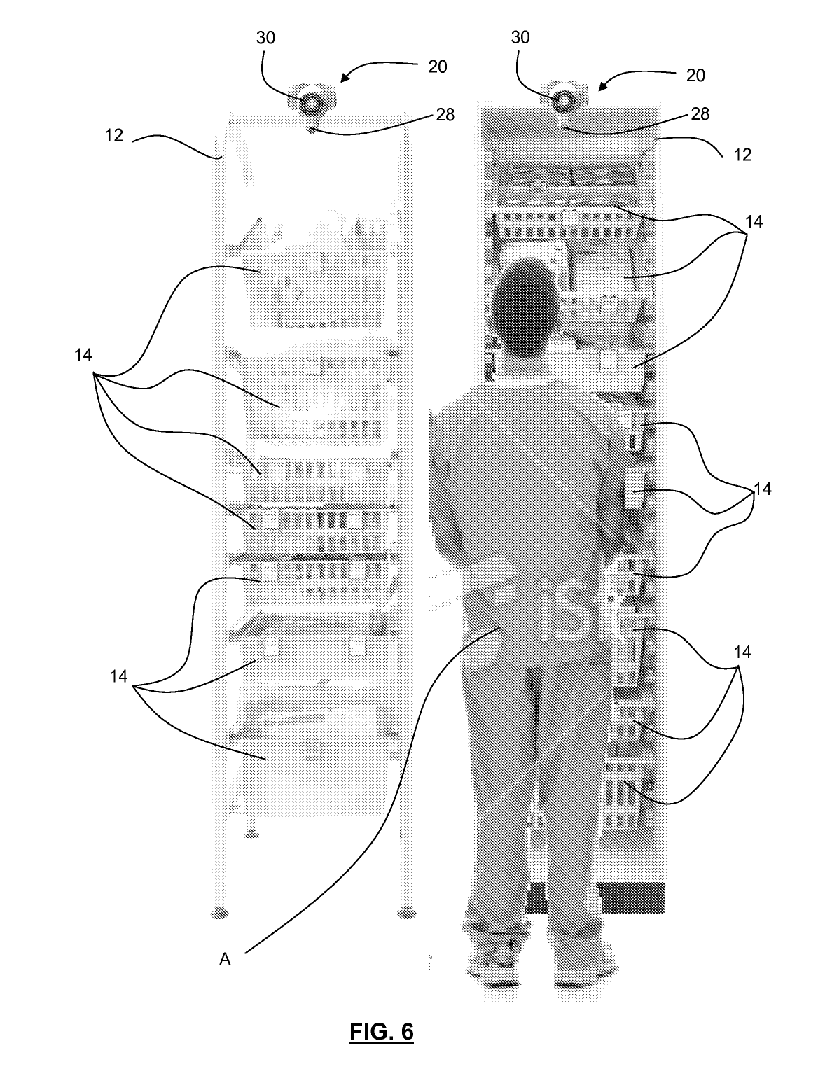

Turning now to FIG. 6, in some embodiments the second camera 30 may be used to enhance operation of the imaging unit 20. For example, at least one of the first and second cameras 22, 30 may be used to capture facial features and/or gestures (such as facial gestures or hand signals) to communicate additional information to the imaging unit 20. This may be particularly useful for associating patient information with particular medical products, which can be important for accurate patient charging, for facial recognition and/or for access control.

For example, as shown in FIG. 6, as a user (indicated generally as A) approaches the storage depot 12, the user A can look at the confirmation light 28 until a particular color is displayed (e.g. green) which indicates that the imaging unit 20 is active.

The user A can then communicate information to the imaging unit 20 for example by making a particular facial gesture (e.g. a smile) or a hand signal (e.g. holding up two fingers). This may be done to indicate that a specific task (e.g. replenishment) will be performed, or reference a particular patient (e.g. by patient number, bed number, and so on.) to link one or more picked medical products thereto. This can be useful for charge capturing to ensure that the system 100 knows which user picked which medical products, what particular user those medical products where used for, and what (if any) of those medical products were returned to the storage depot 12 after the medical treatment.

In some embodiments, once the desired medical products have been picked and captured by the imaging unit 20, the user A may make another gesture to terminate the activity, deactivate the imaging unit 20, and so on.

In some embodiments, information on what the system 100 detected is displayed on a display screen (e.g. an LCD).

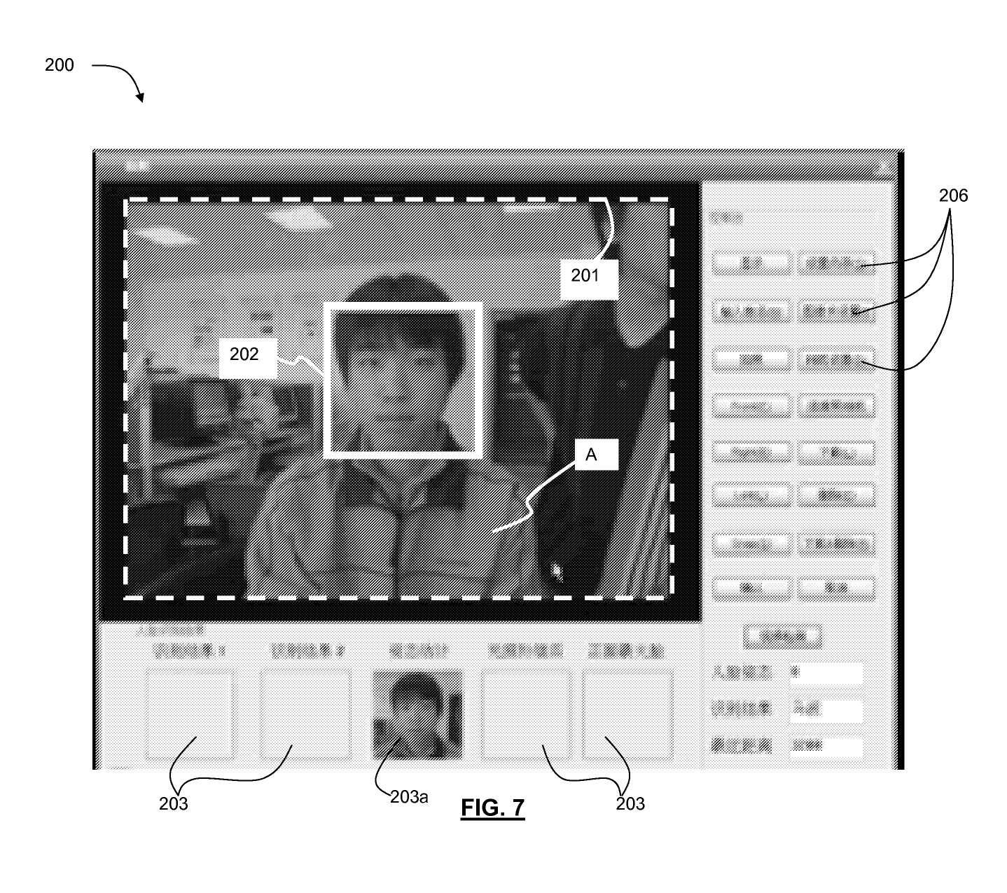

For example, with reference to the screenshot 200 shown in FIG. 7, the imaging unit 20 may observe an image 201 of the room in front of the storage depot 12 using the second camera 30. When the user A is detected, the imaging unit 20 can activate the confirmation light 28 (shown in FIG. 6) to inform the user A that the imaging unit 20 is active and awaiting instructions.

The user's face may be identified generally within a detection zone 202. In some embodiments, the user's face in the detection zone 202 can then be compared to faces of authorized users to help with identification of the user A (e.g. confirming that the user A is in fact authorized to use the storage depot 12). The user's face may also be compared to previously identified facial gestures 203 (such as facial gesture 203a) to determine whether the user A is trying to initiate a particular activity (e.g. picking a medical product).

In some embodiments, one or more control buttons 206 may be presented during a training sequence to store particular user faces 203 and/or facial gestures for subsequent reference. For example, images of authorized users could be captured during a training mode and stored in the database 103.

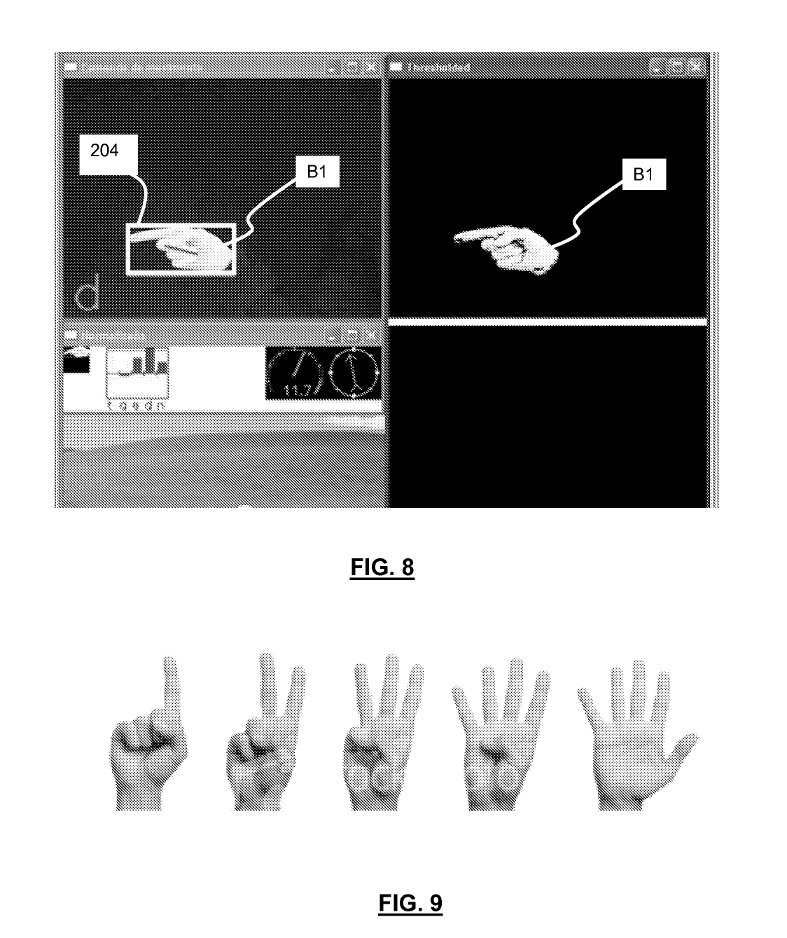

Turning now to FIG. 8, in some embodiments a user's hand (indicated generally as B) may be used to control or provide input to the imaging unit 20. For example, as shown the user's first hand B1 has been located (e.g. using one or more of the cameras 22, 30) and has been identified generally within a detection zone 204. The movement and/or shape of the hand B1 can then be tracked using the detection zone 204 to determine whether the user is trying to provide particular instructions to the imaging unit using hand signals.

FIG. 9 shows various examples of hand signals that could be used to communicate with the imaging unit 20. For example, a particular number of fingers can be held up as different signals. In other embodiments, dynamic hand gestures (e.g. swipes, circular motions, etc.) may be used as signals. Using hand signals in this manner can be beneficial as it can allow the imaging unit 20 to be controlled generally without the user A physically touching an input device. This can help avoid contamination and encourage a sterile environment.

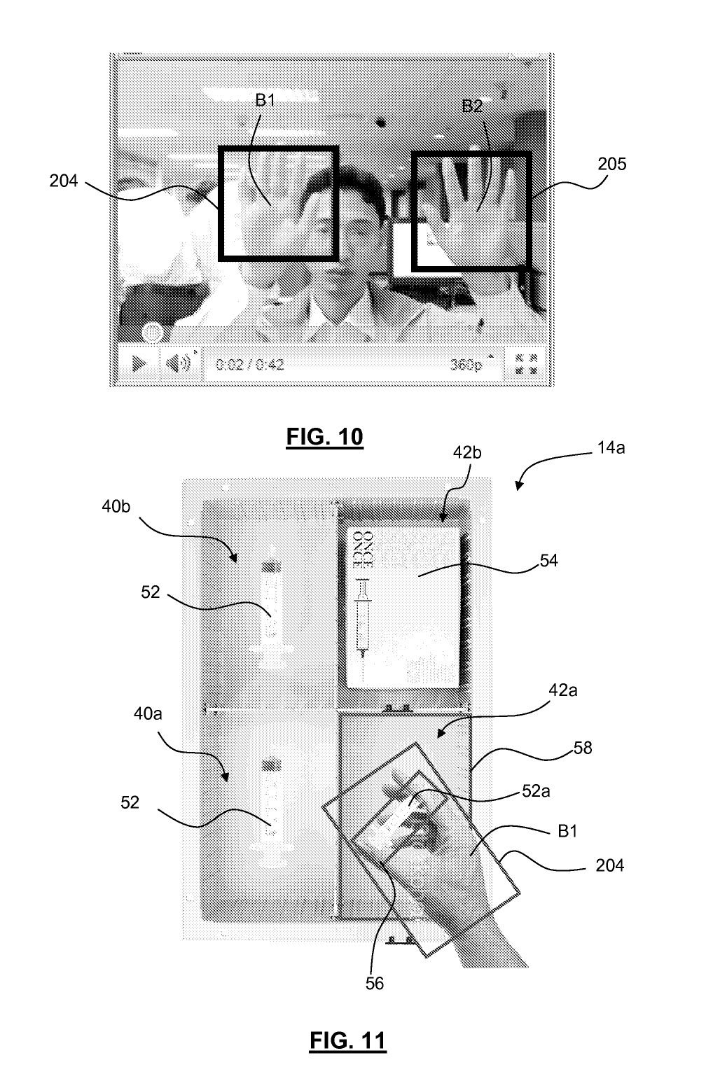

As shown in FIG. 10, in some embodiments, two hands (B1 and B2) may be detected by the second camera 30 and tracked in two detection zones 204, 205. This may allow for a greater number of commands and/or information to be communicated to the imaging unit 20. For example, the hand signals of the hands B1, B2 can be used to link picked medical products to a particular patient (e.g. by using the hands B1, B2 to give the patient number, indicate a bed number, or using other techniques).

Turning now to FIG. 11, the user's hand B1 can also be detected by the first camera 22 when removing a particular medical product 52a from a storage compartment (e.g. the first drawer 14a). In particular, the detection zone 204 can track the movement of the first hand B1 (e.g. speed, direction, etc.) using one or more of the cameras 22, 30. This movement information can assist the imaging unit 20 in determining which medical products have been picked.

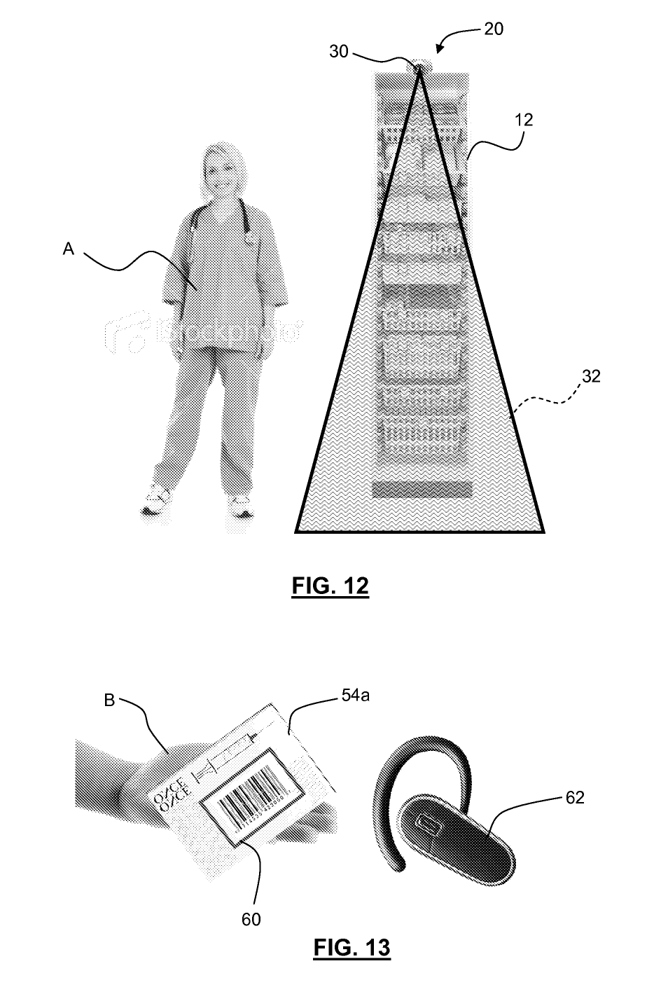

Turning now to FIG. 12, in some embodiments the imaging unit 20 can be adapted to be active when a user A is within the second detection region 32, but inactive when the user A is positioned beyond the second detection region 32. For instance, the user A can step into the second detection region 32 for a particular amount of time (e.g. a time delay of three seconds) until the confirmation light 28 indicates that the imaging unit 20 is active. This time delay can allow the imaging unit 20 to be used in areas with significant amounts of traffic while reducing the potential for false activations.

Once the user A has completed their tasks, they can simply step out of the second detection region 32, deactivating the imaging unit 20.

Turning now to FIG. 13, as shown a medical product 54a may have a bar code 60 thereon. During use (e.g. when replenishing stock), the user's hand B may present the medical product 54a to the camera 22 so that the bar code 60 is visible. In some embodiments, the confirmation light 28 may indicate that the bar code 60 has been recognized (e.g. by flashing green) and the medical product 54a may then be placed in one of the storage compartments 14. The bar code 60 may also be used to communicate other information to the imaging unit 20 (e.g. expiry dates, serial and lot numbers, etc.).

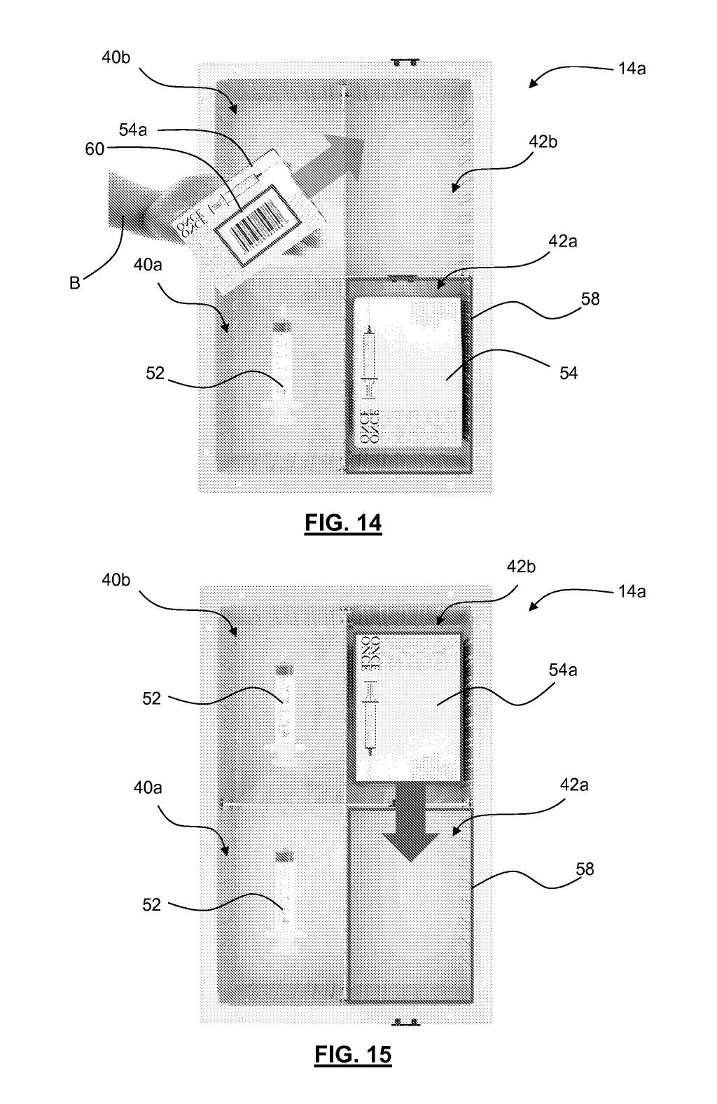

In some embodiments, the imaging unit 20 may inform the user about which storage compartment 14 is the proper one for replenishment. For example, the user may be wearing a wireless communication device 62 (e.g. a Bluetooth headset) or carrying a personal data assistant or smartphone. Once the bar code 60 of the medical product 54a has been recognized by the imaging unit 20, a computerized voice may then be used to guide the user to the proper storage compartment 14 within the storage depot 12, for example using the wireless communication device 62. For example, as shown in FIG. 14, the user may be directed to place the medical product 54a in the right-hand secondary region 42b of the first drawer 14a.

In some embodiments, the imagining unit 20 may inform the user of the proper storage compartment in other ways, which may be audible (e.g. a wired or wireless speaker), visual (e.g. using a display such as an LCD display, or by activating a light on a particular storage compartment 12), and so on.

In some embodiments, as shown in FIG. 15, once a primary storage region 42a is empty, medical products from the corresponding secondary storage region 42b can be rotated or moved into the primary storage region 42a.

In some embodiments, the imagining unit 20 may prompt a user to rotate or move the medical product to the primary storage region 42a.

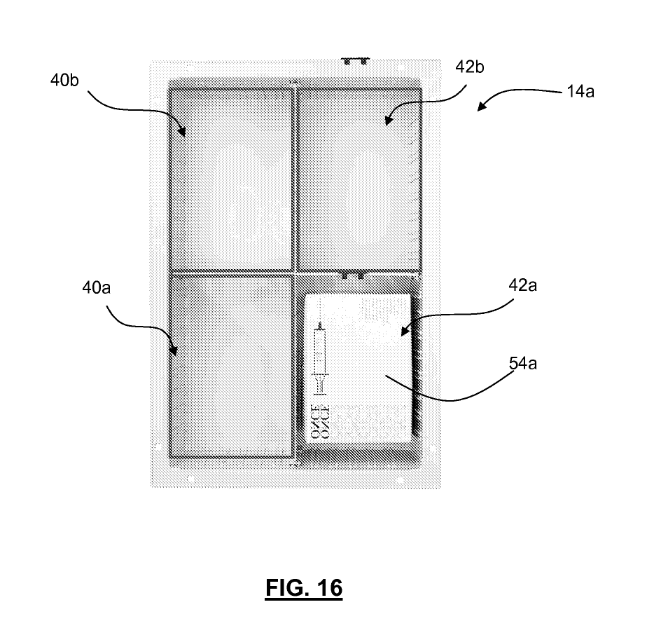

Turning now to FIG. 16, in some embodiments the imaging unit 20 may be adapted to trigger a resupply request when one or more secondary storage regions 40b, 42b are determined to be empty.

While the present apparatus, systems and methods for tracking have been shown and described with reference to different aspects thereof, it will be recognized by those skilled in the art that various changes in form and detail may be made herein without departing from the scope as defined by the appended claims.

1. An apparatus for tracking medical products, comprising:
a storage depot having a plurality of storage compartments;
an imaging unit having a first camera adapted to observe the storage compartments to track medical products therein, the first camera mounted at a top end of the storage depot and orientated downwards so as to define a detection region for observing each storage compartment; and
a second camera orientated outwardly to define a second detection region, adapted for detecting the presence or absence of a user within the second detection region for controlling the imaging unit;
wherein the imaging unit is adapted to determine whether a particular storage compartment is empty without necessarily identifying a type of medical product therein.

2. The apparatus of claim 1, wherein at least one of shape, size and pattern recognition algorithms are used to visually identify particular medical products in one or more regions of the storage compartments.

3. The apparatus of claim 1, wherein the second camera is adapted to detect facial gestures of the user within the second detection region for controlling the imaging unit.

4. The apparatus of claim 1, wherein the second camera is adapted to detect facial gestures of the user within the second detection region for controlling the imaging unit.

5. An apparatus for tracking medical products, comprising:
a storage depot having a plurality of storage compartments; and
an imaging unit having a first camera adapted to observe the storage compartments to track medical products therein.

6. The apparatus of claim 5, wherein the first camera is mounted at a top end of the storage depot and is orientated downwards so as to define a detection region for observing each storage compartment.

7. The apparatus of claim 5, wherein the imaging unit is adapted to determine whether a particular storage compartment is empty without identifying a type of medical product therein.

8. The apparatus of claim 5, wherein at least one of shape, size and pattern recognition algorithms are used to visually identify particular medical products in one or more regions of the storage compartments.

9. The apparatus of claim 5, further comprising a second camera orientated outwardly to define a second detection region.

10. The apparatus of claim 9, wherein the second camera is adapted for detecting the presence or absence of a user within the second detection region.

11. The apparatus of claim 9 wherein the second camera is adapted to detect facial gestures of the user within the second detection region.

12. The apparatus of claim 5, wherein the imaging unit is adapted to read optical markings associated with one or more medical products in the storage compartments.

13. The apparatus of claim 5, wherein the imaging unit includes an imaging processor for analyzing image data to generate processed data.

14. The apparatus of claim 13, wherein the imaging processor is adapted to send the processed data to a server.

15. The apparatus of claim 14, wherein the imaging processor is adapted to determine a probability of certainty for the processed data sent to the server.

16. The apparatus of claim 5, wherein the imaging unit is adapted to send raw image data to an image processing server.

17. The apparatus of claim 5, wherein the imaging unit is adapted to learn a particular layout of each storage compartment.

18. A system for tracking medical products, comprising:
at least one server;
at least one storage depot, each storage depot having a plurality of storage compartments; and
at least one imaging unit having a first camera adapted to observe the storage compartments of the at least one storage depot to track medical products therein;
wherein the imaging unit is adapted to communicate data to the at least one server.

19. The system of claim 18, wherein each imaging unit includes an imaging processor for analyzing image data captured by the first camera, and the imaging processor is adapted to send processed data to the server.

20. The system of claim 18, further comprising a wireless communication device, and wherein the at least one imaging unit is adapted to generate a computerized voice to guide a user to a particular storage compartment.
